(19) United States
(12) Patent Application Publication
(10) Pub. No.: US 2023/0181060 A1
(43) Pub. Date: Jun. 15, 2023

(54) MEASURING METHOD AND DEVICE

(71) Applicant: Koneksa Health Inc., New York, NY (US)

(72) Inventors: Peter John KELLY, London (GB); Robert Davis ELLIS, New York, NY (US)

(21) Appl. No.: 17/549,226

(22) Filed: Dec. 13, 2021

(51) Int. Cl. A61B 5/11; A61B 5/00

(52) U.S. Cl. CPC A61B 5/1121; A61B 5/7225; A61B 5/1126; A61B 2562/0219

(57) ABSTRACT

A method and apparatus are for determining a medical biomarker indicative of a neurodegenerative disease. The method includes receiving motion data from a sensor held or mounted for rotation with a body part of the user during a repeated rotational motion of the body part. The motion data are provided relative to a frame of reference associated with the sensor. The motion data are processed to identify orientation in the frame of reference associated with the sensor of primary, secondary, and tertiary axes of rotation for the motion of the body part of the user, each of the primary, secondary and tertiary axes being mutually perpendicular to one another. A medical biomarker is extracted by analysing the rotational motion of the body part of the user about the primary, secondary and tertiary axes.

### FIELD OF THE INVENTION

The present invention relates to the field of measurement of rotational movement of a user. The invention has particular although not exclusive relevance to methods and devices for measuring and/or analysing the movement of a user to analyse the pronation and supination movements periods of a user.

### BACKGROUND

Accelerometers and/or gyroscopes are commonly and readily available today. For example, most modern mobile telephones are supplied with accelerometers and/or gyroscopes integral to the telephone. The output from these sensors may be analysed to determine features associated with the motion and orientation of the mobile telephone. Typically this data processing is directed to assist in navigation of a user or towards providing input for applications such as games, but may also be used in fitness tracker apps, such as step counters, etc.

Current uses are typically focused on the leisure market—where accuracy is less important than repeatability. The devices may be dedicated devices designed to monitor the motion of the device by considering user input in the form of physical motions applied to the device or they may take the form of a software application running on a user device such as a mobile (cellular) telephone or a smart watch or the like. In most use cases, the processing of the input data is simplified to achieve a “good enough” result to identify e.g. an input rotation axis about which the user intends to rotate the device. Deeper complexity in processing, in order to extract information pertaining to rotations about multiple axes is rarely used, with many applications preferring to identify rotations about a substantially invariant rotation axis in the frame of reference of the measuring device.

In addition, it may be desirable to use motion sensing to monitor movements when measuring the movement of a body part, for example as part of a medical assessment. Prior art devices usually assume that the accelerometers within the accelerometry device are aligned with the axis of rotation of the part of the body being monitored, requiring that the accelerometry device be carefully positioned at the start of the assessment and secured in place such that it does not move during the assessment.

This invention does not require the device to be aligned along any particular axis, or that the axis of rotation needs to be known ahead of time. This invention eliminates the requirement that a trained clinician or other expert correctly position the device and then actively monitor the subject and the device to prevent changes in positioning during the assessment. This invention provides more accurate data more consistently, significantly improving decision-making based on these health measures.

The dataset provided by such sensors contains a wealth of useful information, and the inventors have identified various needs which are unfulfilled in the existing art. An example is that a need exists for more nuanced measurements as well as a more detailed analysis of those measurements.

Moreover, existing algorithms might have difficulty determining the start time of the first rotation and the end time of the last rotation, resulting in errors in the turn rate and other feature scores calculated. This invention significantly reduces those errors.

Some medical conditions, such as neurodegenerative diseases, central nervous system disorders and other diseases impacting musculature control, may result in a subject having an imperfect ability to recreate a model motion, so devices and algorithms which assume a specific motion to simplify the analysis may be inappropriate.

Some subjects may be unable or unwilling to wear a device in a specific position (e.g. wrist) or a specific device (e.g. an ankle tag watch), so the device and algorithm should ideally be agnostic as regards to wear position and should be valid across a variety of hardware devices to accommodate these difficulties.

### SUMMARY

Aspects of the invention are set out in the independent claims and preferred features are set out in the dependent claims.

According to a first aspect there is provided a method of determining a medical biomarker indicative of a neurodegenerative disease or other disease impacting musculature control. The method includes receiving motion data from a sensor held or mounted for rotation with a body part of the user during a repeated rotational motion of the body part. The motion data are provided relative to a frame of reference associated with the sensor. The motion data are then processed to identify the orientation in the frame of reference associated with the sensor of primary, secondary, and tertiary axes of rotation for the motion of the body part of the user, each of the primary, secondary and tertiary axes being mutually perpendicular to one another. A medical biomarker is extracted by analysing the rotational motion of the body part of the user about at least one of the primary, secondary and tertiary axes. That is to say that the analysis of the rotational motion which is received relating to the primary, secondary and tertiary axes is analysed to extract the medical biomarker. For the avoidance of doubt, the analysis itself may make use of the data relating to all three axes, just two of the axes, or just one axis in some cases. The motion data may take the form of rotational or linear position, velocity or acceleration information along or around each of three axes.

The analysing of the rotational motion of the body part of the user may be performed in a continuous or ongoing manner throughout the repeated motion. In other words, the analysis may occur throughout a continuous period, in which the rotational motion is performed repeatedly.

Analysing the rotational motion may include extracting the rotational motion along the first, second and/or third axes as a function of time.

The method may further comprise extracting local maxima and minima by identifying times at which peaks and troughs have a prominence having a magnitude greater than a predetermined threshold.

The analysing may be performed only on motions having a prominence greater than the predetermined threshold.

The method may be executed such that the first and/or last motion is excluded from the analysing. In further examples the first M and/or final N motions may be excluded from the analysis. M and N may be different integers or the same integer.

The medical biomarker may be indicative of a discrepancy between the motion of the body part and a model motion which the user is instructed to make.

Extracting the medical biomarker may include extracting one or more of: frequency of repetition of the rotational motion; time for a single rotational motion; average time for a single rotational motion; an angular amplitude of the rotational motion; asymmetry of an angular amplitude of the rotational motion relative to a neutral point; asymmetry of angular velocity or acceleration of the rotational movement; and/or a ratio of an angular amplitude of the rotational motion along one of the first second and third axes to an angular amplitude of the rotational motion along a different one of the first, second and third axes.

Extracting the medical biomarker may include detecting a change in one or more of: frequency of repetition of the rotational motion; time for a single rotational motion; the orientation of one or more of the first, second and third axes in the frame of reference associated with the sensor; an angular amplitude of the rotational motion; asymmetry of an angular amplitude of the rotational motion relative to a neutral point; asymmetry of angular velocity or acceleration of the rotational movement; and/or a ratio of an angular amplitude of the rotational motion along one of the first second and third axes to an angular amplitude of the rotational motion along a different one of the first, second and third axes.

In some cases a time period for which the repeated rotational motion occurs is divided into two or more distinct sub-periods of time and wherein the analysing is performed individually on motion data received during one of the sub-periods of time. In further examples the analysing may be performed on more than one of the sub-periods of time independently of each other. In yet further examples each of the sub-periods of time may be analysed independently of all the other sub-periods of time.

Optionally the processing uses principal component analysis to identify the orientation of the first, second and third axes.

A low-pass filter may be applied to the motion data prior to the identifying step being performed.

The repeated rotational motion may be a pronation and supination of the hand or bending and straightening of a knee or elbow.

The method may further comprise calculating, based on the orientation of the primary, secondary and tertiary axes, a transform to align the received motion data with the primary, secondary and tertiary axes. Optionally the transform is applied to the received motion data to extract the rotational motion of the user along each of the primary, secondary and tertiary axes.

The invention also provides a tangible computer readable medium comprising computer implementable instructions for causing a programmable computer device to carry out the steps of the method described above.

The invention also provides an apparatus comprising one or more processors and memory. The apparatus is arranged to determine a medical biomarker indicative of a neurodegenerative disease. The apparatus is arranged to receive motion data from a sensor held or mounted for rotation with the body part of a user during a repeated rotational motion of the body part. The motion data is provided relative to a frame of reference associated with the sensor and the apparatus is arranged to process the data to identify the orientation in the frame of reference associated with the sensor of primary, secondary, and tertiary axes of rotation for the motion of the body part of the user. Each of the primary, secondary and tertiary axes are mutually perpendicular to one another. The apparatus is further arranged to extract the medical biomarker by analysing the rotational motion of the body part of the user about the primary, secondary and tertiary axes. That is to say that the analysis of the rotational motion which is received relating to the primary, secondary and tertiary axes is analysed to extract the medical biomarker. For the avoidance of doubt, the analysis itself may make use of the data relating to all three axes, just two of the axes, or just one axis in some cases. The motion data may take the form of rotational or linear position, velocity or acceleration information along or around each of three axes.

The apparatus may further include an accelerometer and/or a gyroscope for supplying the motion data.

The one or more processors and memory may be further configured to analyse the rotational motion of the body part of the user is performed in a continuous manner throughout the repeated motion.

The one or more processors and memory is further configured to analyse the rotational motion to extract the rotational motion along the first, second and/or third axes as a function of time.

The one or more processors and memory may be further configured to extract local maxima and minima by identifying times at which peaks and troughs have a prominence having a magnitude greater than a predetermined threshold.

The one or more processors and memory may be further configured to analyse only motions having a prominence greater than the predetermined threshold.

The one or more processors and memory may be further configured to exclude the first and/or last motion from the analysis. In further examples the first M and/or final N motions may be excluded from the analysis. M and N may be different integers or the same integer.

The medical biomarker may be indicative of a discrepancy between the motion of the body part and a model motion which the user is instructed to make.

Optionally, as part of extracting the medical biomarker, the one or more processors and memory is further configured to extract one or more of: frequency of repletion of the rotational motion; time for a single rotational motion; average time for a single rotational motion; an angular amplitude of the rotational motion; asymmetry of an angular amplitude of the rotational motion relative to a neutral point; asymmetry of angular velocity or so acceleration of the rotational movement; and/or a ratio of an angular amplitude of the rotational motion along one of the first second and third axes to an angular amplitude of the rotational motion along a different one of the first, second and third axes.

Optionally, as part of extracting the medical biomarker, the one or more processors and memory is further configured to detect a change in one or more of: frequency of repletion of the rotational motion; time for a single rotational motion; the orientation of one or more of the first, second and third axes in the frame of reference associated with the sensor; an angular amplitude of the rotational motion; asymmetry of an angular amplitude of the rotational motion relative to a neutral point; asymmetry of angular velocity or acceleration of the rotational movement; and/or a ratio of an angular amplitude of the rotational motion along one of the first second and third axes to an angular amplitude of the rotational motion along a different one of the first, second and third axes.

The one or more processors and memory may be further configured to divide a time period for which the repeated rotational motion occurs into two or more distinct sub-periods of time and to perform the analysing individually on motion data received during one of the sub-periods of time. In further examples the analysing may be performed on more than one of the sub-periods of time independently of each other. In yet further examples each of the sub-periods of time may be analysed independently of all the other sub-periods of time.

The one or more processors and memory may be further configured to use principal component analysis to identify the orientation of the first, second and third axes.

The one or more processors and memory may be further configured to apply a low-pass filter to the motion data prior to the performing the identifying.

The repeated rotational motion may be a pronation and supination of the hand or bending and straightening of a knee or elbow.

The one or more processors and memory may be further configured to calculate, based on the orientation of the primary, secondary and tertiary axes, a transform to align the received motion data with the primary, secondary and tertiary axes.

The one or more processors and memory may be further configured to apply the transform to the received motion data to extract the rotational motion of the user along each of the primary, secondary and tertiary axes.

The one or more processors and the memory may be provided on a first device and wherein the gyroscope and/or accelerometer is provided on a second device located in a remote location relative to the first device; and wherein the first and second devices include cooperating communications units for communicating with one another.

The invention also provides an apparatus as described above forming part of a clinical trial system comprising a central computer that communicates with a plurality of user devices. Each user device is arranged to collect motion data relating to repeated rotational motion of a body part of a user associated with the user device. The central computer or at least one user device comprises the apparatus described above for determining a medical biomarker.

The analysis may be performed locally, or centrally at the central computer.

Optionally, the biomarker includes an axial variability parameter, Vₐₓ defined as:

$${V}_{ax} = 1 - \frac{max \{ {v}_{1} , {v}_{2} , {v}_{3} \}}{{\sum }_{i = 1}^{3} {v}_{i}}$$

in which v₁, v₂, and v₃ are the eigenvalues of the data about the primary, secondary and tertiary axes respectively.

Moreover, in some cases, extracting the biomarker further includes comparing the biomarker to thresholds derived from statistical analyses of the biomarker across a population.

As an example the biomarker may be the biomarker, Vₐₓ, although the general principles set out above in which a biomarker is extracted and compared to a threshold apply equally to analysis of any biomarker extracted from the rotational motion data. As noted above, the threshold may be extracted from statistical analyses of that biomarker (or indeed of other, related biomarkers) across a population, for example examining correlations between the value for the biomarker and independent assessments of e.g. the severity of a particular disease or disorder.

The invention also provides a tangible computer readable medium comprising computer implementable instructions for causing a programmable computer device to become configured as an apparatus according to the apparatuses described above.

### BRIEF DESCRIPTION OF THE DRAWINGS

Exemplary embodiments of the invention will now be described with reference to the accompanying figures in which:

FIG. 1A schematically illustrates a clinical trial in which the movement of users taking part in the trial is determined by user devices worn or carried by the users and reported to a central server for collection and analysis;

FIG. 1B is a block diagram illustrating the main electronic parts of the system shown in FIG. 1A;

FIG. 2 is a block diagram illustrating the main components of a user device shown in FIG. 1B,

FIG. 3 is a schematic diagram illustrating an example motion which the system or method is arranged to assess;

FIG. 4 is a flow diagram illustrating steps in an example method for extracting a medical biomarker from rotational motion data;

FIG. 5A is a plot illustrating raw rotational velocity data along three orthogonal axes received from a sensor during a user attempting to make a model motion;

FIG. 5B is a plot illustrating the rotational velocity data of FIG. 5A after processing to align the receive motion data with primary, secondary and tertiary axes of rotation;

FIG. 5C is a plot illustrating rotation angle extracted from the rotational velocity data of FIG. 5B;

FIG. 6 illustrates plot of rotational angle for a set of three half-rotations; and

FIGS. 7A and 7B illustrate data collected of a biomarker relating to axial variability in healthy and diseased populations.

In the drawings, like reference numerals are used to indicate like elements.

### DETAILED DESCRIPTION

Overview

As summarised above, the invention provides alternative ways for analysing a user's movements. The methods and devices provided by the invention can be used in various applications, such as in fitness trackers and the like. However, the invention can also be used in a medical setting which will now be described.

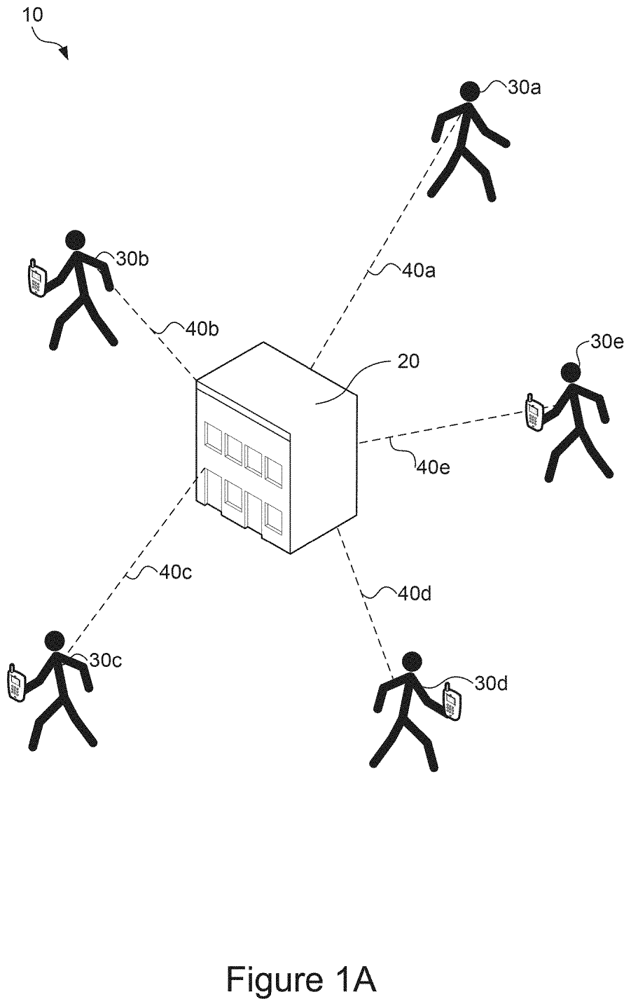

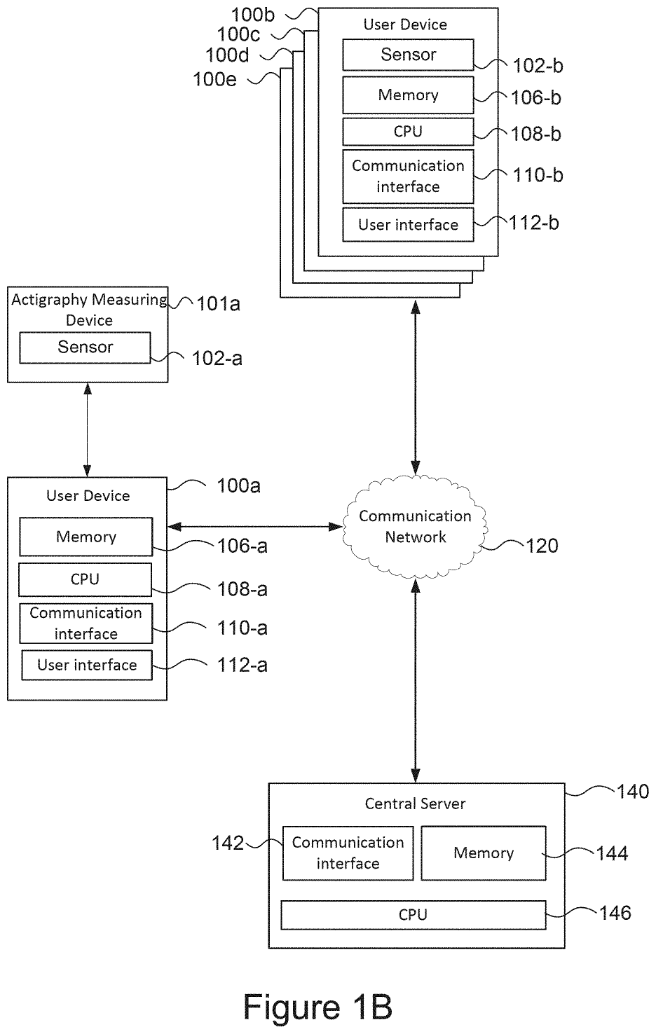

More specifically, FIGS. 1A and 1B illustrate how the invention can be used in a clinical trial system 10 in which a number of subjects (also referred to below as users) 30a to 30e use a respective user device 100a to 100e to monitor the movements of the corresponding subject when they are attempting to make a model motion comprising a rotational movement of a body part. In many examples, the model motion comprises a set of repetitions of a particular motion, for example 10, 20, 50 or even 100 repetitions. The motion may for example be a pronation/supination motion of the wrist or bending and straightening of a knee or elbow. The information gathered by the user devices 100 is transmitted over a communication network 120 (represented in FIG. 1A by the broken lines 40a to 40e) back to a central server 140 with results that may be displayed within the clinic 20.

The clinic 20 may be a health centre such as a hospital or doctor's surgery. It may comprise a single centre or a number of centres located in a number of different geographical locations. The subjects 30a-30e are patients of the clinic 20 and are taking part in a medical trial, organised by the clinic 20. Each of the patients in the medical trial are cohorted into groups with the same medical condition.

Each of the subjects 30a-30e is provided with a user device 100 that may be dedicated to the clinic and returned to the clinic after the trial is over. Alternatively, the clinic may provide the subject with a software application that they can run on their own user device—such as a cellular telephone or a smart watch or the like. In either case, each subject is asked to wear or carry their user device so that a sensor associated with the user device can capture the movements of the user during the clinical trial. As shown in FIG. 1B, some user devices 100 have a built-in sensor 102 but some (in this example user device 100a) do not. Where the user device 100 does not have a sensor, a separate actigraphy measuring device 101 is provided that has a sensor 102-a for capturing the movements of the subject 30a. The sensor 102, 102-a is arranged to be held by the user, or otherwise mounted for rotation with the body part the motion of which is to be assessed. The user's motions in trying to make a model motion are thereby captured by the sensor.

As discussed below in more detail, the sensor 102, 102-a may be one or more accelerometers and/or gyroscopes. Accelerometers typically provide linear acceleration information in one, two or three orthogonal directions which depend on the orientation of the accelerometer. Gyroscopes typically provide angular velocity information in one, two or three orthogonal directions. With appropriate analysis, and use of boundary conditions (e.g., constants of integration), it is possible to convert between acceleration, velocity and position (or between angular acceleration, angular velocity and orientation) by integrating or differentiating the data as appropriate. By making further assumptions about the absolute orientation of the device (which can be extracted using the direction of the local gravitational and/or magnetic fields and applying location-specific corrections derivable from GPS coordinates), it may be possible in some cases to convert between linear acceleration and angular velocity, if certain assumptions can be made. By analysing the sensor data, the user device 100 can determine rotational movement information about the body part of the subject which is then transmitted (wirelessly or over a wired connection) as subject data to the central server 140 for further analysis as part of the medical trial.

In one example, the subject data provided to the central server 140 comprises rotational data and identification data that identifies the subject to which the rotational motion data relates. The rotational data may comprise absolute and/or changes in one or more of: frequency of repetition of the rotational motion; time for a single rotational motion; the orientation of one or more of the first, second and third axes in the frame of reference associated with the sensor; an angular amplitude of the rotational motion; and/or a ratio of an angular amplitude of the rotational motion along one of the first second and third axes to an angular amplitude of the rotational motion along a different one of the first, second and third axes, over a period specified by the trial, for example a day, week, month or year. The subject data may be retrieved from the user device 100 when the subject visits the clinic, or the subject data may be transmitted to the clinic over a cellular or wired telephone or computer network (wirelessly or over a wired connection). Subject data collected at the clinic can be supplemented with physical observations and tests which can only be done at the clinic 20 and not monitored remotely. Accuracy of the data provided to the clinic 20 about a subject's activity outside of the clinic 20 and at home is important in ensuring that the medical trial receives a true representation of the subject's activity during the monitored period. This can help to determine the efficacy of the clinical trial's therapies.

In another example, the subject data provided to the central server 140 comprises the identification data for the subject together with the sensor data, so that the central server 140 processes the sensor data for each subject from which the central server 140 analyses the rotational motion data for each subject itself. Although not illustrated in FIG. 1B, in this case, the central server 140 further comprises a user interface including a user input device such as a keyboard, and/or software for processing the data collected from the user devices of the system.

The subject data may be indicative of various aspects of physical health of a user. At a very high level, the amount of time a user is able to make a specific motion for, or the number or speed of those motions may be indicative of a general level of physical fitness. By taking a more nuanced view, additional information may be obtained. In some cases, the high level data may be indicative of a need for a patient to be called into the clinic or could indicate that the patient may be required to spend a short amount of time in hospital. In some examples, the collected patient data may be used by the clinic to help book appointments for the patient with a doctor or clinician as required.

The subject data can also be used for athletic performance measurement and management. Detailed analysis of range and/pr speed of motion during targeted assessments of athletic activities can be provided to the athletes or their trainers and coaches. That data can then be used to inform training regimens to improve athletic performance.

The subject data can also be used for physical therapy performance measurement and management. Detailed analysis of range and/or speed of motion during targeted assessments during managed or unmanaged therapy sessions activities can be provided to the patient or their therapists and doctors. That data can then be used to inform therapeutic regimens to improve recovery programs.

Moreover, the rotational information data derived from a user attempting to replicate a model motion is particularly useful to study in patients known to have or suspected of having one or more medical conditions which are known to affect motion capabilities. In some cases, temporary motion complications may be caused by injury, trauma, inflammation or pain. In other cases, problems with rotational movements such as pronation and supination of a hand or bending and straightening of a knee or elbow can be caused by specific conditions. Some of the conditions which may be particularly important in measuring rotational motion activity include but are not limited to: arthritis, multiple sclerosis (MS), Meniere's disease, brain damage for example caused by a haemorrhage or tumour, Parkinson's disease, orthopaedic surgery on hips or lower body, cancer and associated therapies, cerebral palsy, obesity, gout, muscular dystrophy, stroke, spinal injury, deformities, etc.

The frequency and/or change in frequency during the motion may be extracted by averaging a number of repetitions detected over the time in which those repetitions were detected, or by more sophisticated methods, for example involving Fourier transforms (or other frequency analysis such as a Discrete Cosine Transform) and the like. In whichever manner it is achieved, measurements of frequency or change in frequency during the measurement may be indicative of general physical health in the sense that dexterity generally decreases with age and/or lack of physical fitness. By analysing the exact form of the change in frequency, further information may be derived in which an assessment may be made of the likelihood that a user is developing a particular disease or disorder.

The time taken for a single rotational movement or the average time taken for a single rotational movement and/or a change in those parameters may also be an indication of the general level of fitness of a user since it is related to the speed at which a user can move, which is expected to be lower for elderly or ill users than for young healthy users. In addition, changes in the time (or average time) taken can be indicative of a user tiring out, and the rate at which the user tires out can be indicative of various diseases and/or disorders.

Similarly, the amplitude and/or change in amplitude can be indicative of the general health of a user, since a greater range of movement (tailored to the specific model movement being used) is usually associated with greater physical fitness. In cases where the range of motion changes during the testing, this may be indicative of a user tiring (where the range decreases) or warming up (if the range increases). Either way, the progression of the range of motion over time can be a useful biomarker in assessing a user.

In further examples, a ratio of an angular amplitude of the rotational motion along one of the first second and third axes to an angular amplitude of the rotational motion along a different one of the first, second and third axes and/or a rate of change in such a ratio may be considered. Once more this can be used to assess the ability of the user in making the model motion. Typically, the model motion is one in which a motion is made about a single axis of rotation. As noted below, this axis need not be aligned with one of the three axes along which the sensor measures motion. Nevertheless, by analysing the data which includes rotations about one or more perpendicular axes which are not aligned with the sensor measurement axes, the principal axes of rotation can be identified. By considering the ratio of the magnitude of rotation about the three perpendicular principal axes in this manner, the ability of a user to make a single axis rotation, since the relative magnitude of rotations about secondary and tertiary axes can indicate the degree of compliance with the model motion. As with the other parameters discussed above, the change over time in the ratios calculated in this way can also contain useful information, in the sense that a user may tire or otherwise become less able to accurately replicate the model motion. This may also be indicative of a relevant biomarker for indicating the presence and/or severity of a particular disease and/or disorder. The ratio may be calculated as a ratio of the rotational amplitude, rotational velocity, or energy of rotation about the axes.

The parameters discussed above may be provided as absolute values, or relative to specific thresholds for the model motion (the thresholds being indicative of a typical value in the healthy population). In some cases a user may complete the model motion again at a later date and the performance on subsequent sessions compared to previous sessions (for example using previous sessions to adjust or set the thresholds).

The model motion is usually a repeated motion and continues for a number of repetitions (e.g. 25 repetitions), or for a set time (e.g. 20 seconds). In some cases, the analysis of the motion data may be carried out on the data from the entire duration of the test. In other examples, the motion data may be subdivided into consecutive time periods and each time period analysed separately.

Two or more of the parameters above may be combined together to provide the biomarker. In other words the biomarker may be derived from a combination of various parameters. Combinations such as a weighted average or comparing parameters to a threshold and using the result to navigate a decision tree are examples of applicable methods here.

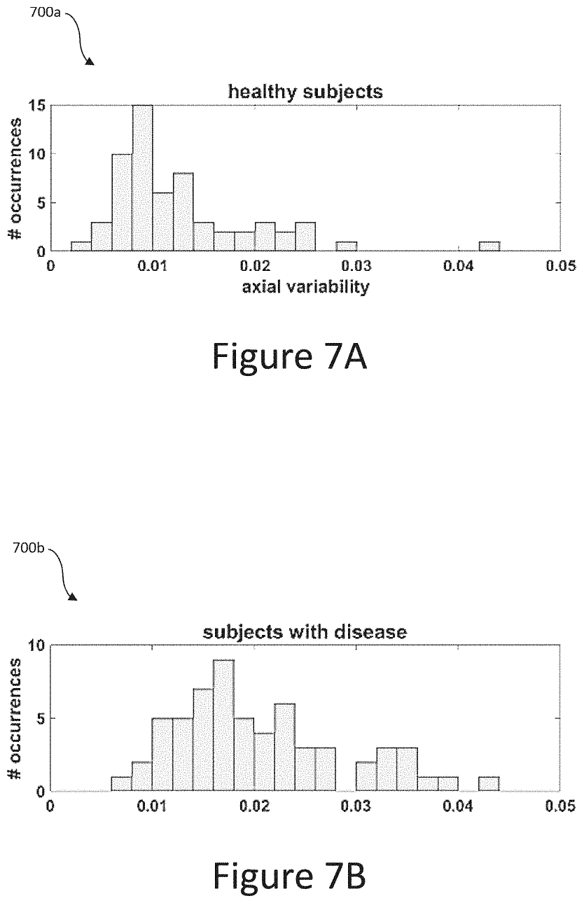

Optionally, the biomarker includes an axial variability parameter, Vₐₓ defined as:

$${V}_{ax} = 1 - \frac{max \{ {v}_{1} , {v}_{2} , {v}_{3} \}}{{\sum }_{i = 1}^{3} {v}_{i}}$$

in which v₁, v₂, and v₃ are the eigenvalues of the data about the primary, secondary and tertiary axes respectively. Moreover, in some cases, extracting the biomarker further includes comparing Vₐₓ to thresholds derived from statistical analyses of Vₐₓ across a population. For example the average (e.g. mean, mode or median value) taken separately across healthy and diseased populations may be calculated and a threshold identified as a value lying in the range of numbers between the average value for each population. This aspect is discussed in more detail below in respect of FIGS. 7A and 7B.

In some examples, combinations of changes may be assessed over time, e.g. on subsequent measurement sessions. For example a subject may initially be able to perform an assessment with stable arm motions having 120 degree motion in each angular direction and be able to complete twenty rotational movements in one minute in their first session. That same patient may subsequently complete a later session with unstable arm motions and only able to move 60 degrees in each angular direction, completing five rotational movements in a minute. This degradation in ability may allow a clinician to determine that the disease has developed in that patient as far as e.g. stage 3 of a recognised grading scale for that particular disease, having started at a less severe grade of e.g. grade 1. Alternatively, a single measurement of these parameters may allow the clinician to identify that this combination of parameters is a 2 on a five point scale.

In yet further cases, the data may be analysed to measure the asymmetry of the rotation about one of the axes from a nominal starting point. Usually this will be assessed about the primary axis, but in some cases, it may be desirable to identify asymmetric motions around the secondary and/or tertiary axes, depending on the specific motion being attempted and the specific disease being assessed. Here the patient may be instructed to hold their body part (e.g. wrist) in a particular way and rotate that body part as far as they can to scope out their range of motion. Once these end points have been identified, the repeated motion can be instructed, and the analysis can proceed as above. In other cases, no calibration may be needed, and the range of motion may be assessed relative to the start angle, under the assumption that the user has started with their body part correctly positioned and oriented.

Similarly, the data may be analysed to identify asymmetry of angular velocity or acceleration of the rotational movement. This operates in much the same manner as set out above in respect of asymmetry in the rotation itself. It is a reasonable assumption that the assessment will start with the user holding the device still, i.e. with angular velocity and angular acceleration equal to zero. Minor motions e.g. due to shaking in some users may be discounted (e.g. filtered out by a low pass filter) or a period of analysis may be deemed to start only when angular velocity and/or angular acceleration about any (or in some cases any two, or all three) of the primary, secondary and tertiary axes exceeds a given threshold. In any event, in whichever manner the asymmetry is extracted from the rotational motion data, the extent to which a user is able to provide a consistent rotational velocity or rotational acceleration motion may be used to indicate the absence, presence and/or severity of a particular disease or disorder.

In yet further cases, the target motion which the user is instructed to make may specify making the motion as asymmetric as possible (in terms of angular motion, angular velocity and/or angular acceleration), and comparing the user's ability to achieve this with results from the population at large to determine a biomarker.

In yet further examples, the measurements taken by the device 100 may be supplemented by other readings taken from the same device (e.g. using an accelerometer to identify walking patterns) or by one or more different, additional devices, for example also carried or held to detect a specific motion. In some cases, the additional device(s) may include sensors to measure or provide one or more of: vital signs, cognition assessments, vocal tremors, etc. of a patient. The biomarker may be formed from a combination of the parameters described above.

Since the data are provided as numerical indications of the various measured parameters, a further advantage of the present invention may be that diagnoses may be provided in a more finely grained manner by converting the measures numerical values into a nuanced diagnosis scheme. For example, many scales for rating the severity of diseases and disorders group patients into wide categories, e.g. a scale of 1 to 5, in which only integer values are used. The present invention can allow a severity value to a much more fine-grained level of detail. This in turn may allow for earlier diagnoses to be made, with a corresponding improvement in prognosis for the patient. For example, taking a crude correspondence between the 1 to 5 scale above (in which 1 indicates no disease or minimum severity and 5 indicates a very advanced stage of the disease or disorder) and a finer-grained percentage scale, it may be seen that a 1 on the coarse scale equates to anywhere in the range of 0% to 20%. Using the coarse scale, the risk would be assessed as minimal and equated to no need to intervene at all. The finer resolution of the present invention, by contrast can be used to indicate that the user is in an early (or very early) stage of the disease or disorder, which would not show up on traditional measures, but nevertheless would benefit from treatment. Early intervention in these cases may lead to better outcomes overall and a great delay in the symptoms progressing to the point that they become debilitating for a user.

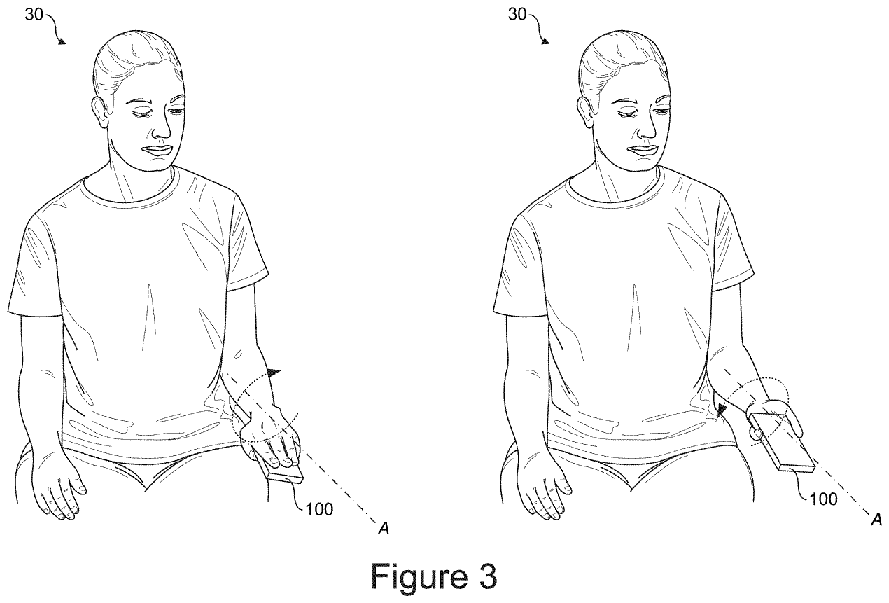

FIG. 3 provides an example of a model motion which a user 30 may be asked to make. Here the user holds the user device 100 in their hand and is asked to make a pronation/supination motion with their wrist. For example, as shown in the left Figure, the user 30 may be asked to hold their arm out horizontally with the back of their hand facing upward in a pronation position and twist their wrist (in the direction indicated in the left Figure) so that the back of their hand faces downwards (this is an initial half turn). This finishing position is a supination position and is shown in the right hand Figure. The motion may be completed by the user returning their hand to the initial position by rotating their hand in the opposite direction (i.e. in the direction of the arrow in the right hand Figure) so that the back of their hand once more faces upwards (this completed motion is a full turn). It is apparent that this motion would cause a rotation about the axis, A, in the Figure.

Although the device 100 is shown as a rectilinear cuboidal device which is held in the users hand to monitor rotational motion (and therefore may be a mobile phone or other personal communications device, or a specially designed device), the device 100 may be any shape. For example, where the device 100 is specially designed for use in this context, it may be ergonomically designed with handles, gripping portions, and so forth. Not only may this provide users with a secure and easy grip on the device 100, but breaking the symmetry of the device in this way may provide clues to simplify the analysis, e.g. by allowing certain assumptions to be made about the general orientation of one or more of the main rotational axes of the assessed motion. In addition, the device 100 may not even be a handheld device, but may be strapped, clipped or otherwise attached to a user's body part, to assess the rotational motion as described herein.

The motion may be repeated a set number of times, or as many times as possible in a given time. In some cases, the starting position and the half-turn position may be switched. In yet further cases, the motion may be a different motion, for example, motions associated with shoulders, knees, hips, and so forth. Where the motion which a user is asked to complete is not one involving the user's hands, the device 100 may be supplied with straps, a holder, or other mounting means to allow the device 100 to be attached to an appropriate part of the user to measure the motion.

Note that the axes along which the sensor 102 provides rotational motion data are fixed relative to the reference frame of the device 100, since the sensor 102 is mounted in the device 100, for example aligned with the rectilinear edges of the device 100 shown in FIG. 3. However, the user may hold the device (or the device may be mounted on the user) in substantially any orientation. Moreover, for reasons of comfort, this orientation may vary not only with mounting location, but from user to user. Overall, this means that the axes about which the device is rotated during the motion (axis A), is not necessarily aligned with the axes along which the sensor 102 provides rotational information. For example, as shown in FIG. 3, the axis A is not aligned with any of the rectilinear edges of the device 100. Of course, where these axes happen to be aligned, the analysis is significantly simpler. Where these axes are not aligned (as in the general case), it is not immediately apparent from the raw data whether a user is actually achieving a single axis rotation or whether there are off-axis components to the motion. Consequently, the processing described below is needed to assess whether and to what extent the motion includes rotations about more than one axis.

In some cases external reference frames may be used to assist in determining the rotational axes. For example, in the example shown in FIG. 3, the user may be instructed to hold their arm out horizontally. If it is known that the intended rotational axis extends horizontally (e.g. is a “roll” axis), then off-axis rotations will look like pitch and yaw motions, which may be detectable by considering the motions in the context of the local gravitational field—since the axis A is intended to be horizontal, deviations from this may be detectable in the local geographic reference frame.

User Device

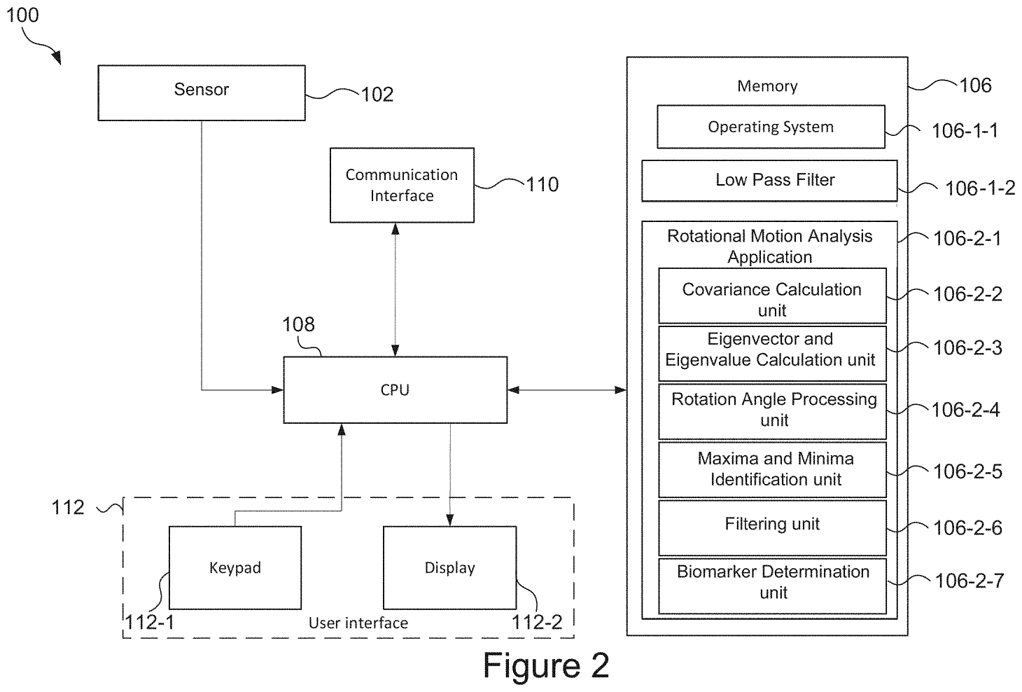

FIG. 2 is a block diagram of a typical user device 100 that is used in the system described above. As shown, in this case, the user device 100 has a sensor 102 that provides rotational motion data to at least one central processing unit (CPU) 108. The operation of the CPU 108 is controlled by software instructions that are stored in memory 106. As shown, the software instructions include an operating system 106-1-1 and a rotational motion analysis application 106-2-1. The motion data from the sensor 102 are processed by the rotational motion analysis application 106-2 to analyse the rotational motion data for the subject.

The user device 100 also includes a communication interface 110 for communicating the subject data determined by the rotational motion analysis application 106-2 to the central server 140; and a user interface 112 comprising a keypad 112-1 and a display 112-2 to allow the subject to interact with the user device 100. The display 112-2 may display one or more icons configured to provide information to the user and/or one or more of: time, date, number of rotations, activity specific icons (model motion, warnings or alerts, etc.), activity duration, reminder messages and/or instructions concerning activity, network connection status, remaining battery power and any other useful information to be displayed to the user.

Sensor Data Analysis

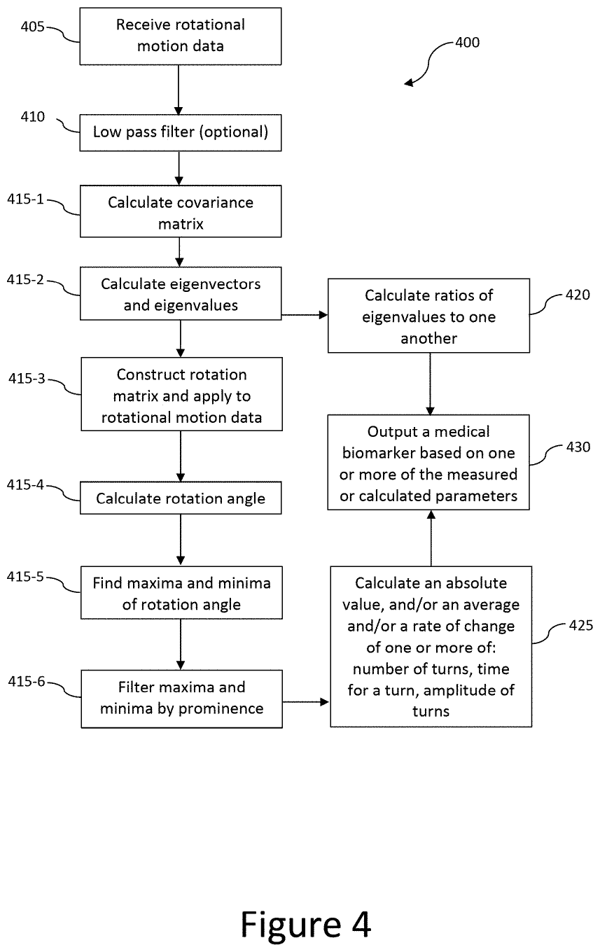

In FIG. 4 a flow chart 400 is shown illustrating the extraction of biomarkers from input rotational motion information. It will be appreciated that there is a correspondence between FIG. 2 and the various operational units 106-1, 106-2 and the steps performed in FIG. 4, and the operation of the operational units 106-1, 106-2 will be discussed below interchangeably with the flow chart shown in FIG. 4.

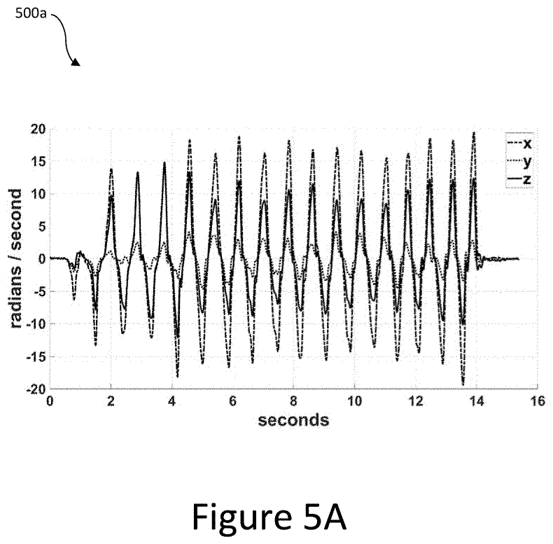

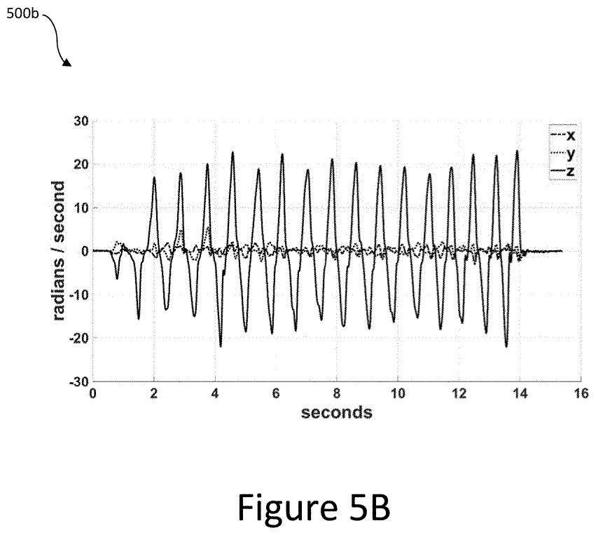

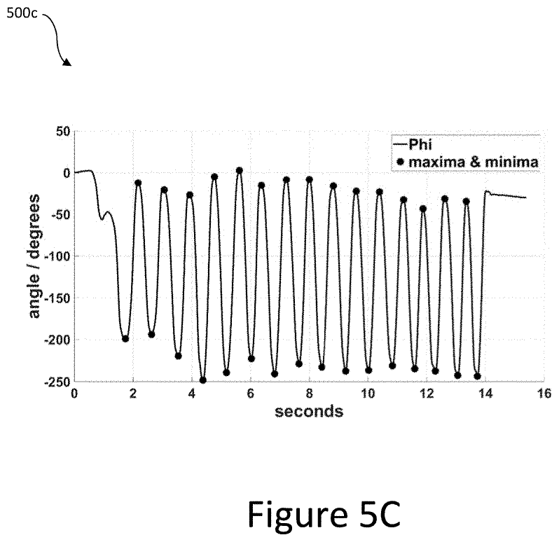

The process starts at step 405 with rotational motion data being received. As noted above, this may take the form of linear or rotational information pertaining to acceleration, velocity or displacement. The sensor registers motion along three orthogonal axes and provides this information as a series of data points at consecutive times. Each data point comprises information in the three axes and may be provided as a vector in the frame of reference of the sensor. An example of this data may be seen in FIG. 5A, discussed in more detail below.

An optional low pass filter 106-2-1 filters the time series measurement data points received from the sensor in step 410, to remove high frequency variations in the accelerometer measurements that are not associated with the rotational movement of the user. The cut off frequency of the low pass filter is typically between 8 Hz and 30 Hz and preferably about 20 Hz.

Subsequently the data are passed to the rotational motion analysis application 106-2. This unit generally performs data processing, with a view to extracting the biomarker information discussed above.

More specifically, in step 415-1, the covariance calculation unit 106-2-2 processes the sensor data to calculate a covariance matrix of the received data. The covariance matrix is a 3×3 matrix having entries calculated from the angular motion around each axis (labelled i or j). In the example below the assumption is made that angular velocity, ωᵢ, are received at each of N times steps, tₖ, as follows:

$${C}_{i , j} = \frac{1}{N} {\sum }_{k = 1}^{N} ( {ω}_{i} ( {t}_{k} ) - \overline{{ω}_{i}} ) ( {ω}_{j} ( {t}_{k} ) - \overline{{ω}_{j}} )$$

in which ω̅ᵢ̅ is the average angular velocity signal around axis i over the span of the (time) duration of the dataset. Since the input measurement is of a repeated motion, including in general rotations in one direction followed by a rotation for the same distance in the opposite direction, the average values are usually small since rotations in one direction are largely cancelled by subsequent rotations in another direction. In cases where the input data is not in the form of angular velocity at the start of the processing, the information can be converted to angular velocity prior to calculating the covariance matrix. In other cases, corresponding covariance matrices can be formed from angular acceleration or displacement data for use in the later processing steps.

Once the covariance matrix has been calculated, the eigenvector and eigenvalue calculation unit 106-2-3 is operable to calculate the eigenvalues and eigenvectors of the covariance matrix, as seen at step 415-2. As shown in step 420, this may also lead to a calculation of the ratio between the eigenvalues along each eigenvector. This is related to the energy or the rotation along each axis, or in other words, the weighting between the rotation along the intended axis of rotation and the non-intended axes of rotation.

Once the eigenvectors and eigenvalues have been extracted from the data, the rotation angle processing unit 106-2-4 is operable to construct, using the eigenvectors, a rotation matrix which rotates the eigenvector having the larges eigenvalue to align with one of the axes of the device, for example the z-axis—see step 415-3. The rotation matrix may also ensure that eigenvector having the next largest eigenvalue aligns with another of the axes of the device, for example the y-axis. This leaves the smallest-eigenvalued eigenvector pointing along the x-axis. Once the rotation matrix has been calculated, the rotation may be applied to the data to align it with the axes of measurement. The result of this applied to the data shown in FIG. 5A may be seen in FIG. 5B, discussed in more detail below.

Other implementations can be used to find the rotation that results in the energy being concentrated in one axis. For example, in another implementation, a ‘brute force’ method whereby multiple rotations are applied and the one that gives the best concentration is selected. Alternatively, this may be solved as an optimisation problem, for which numerous methods are possible e.g. gradient descent.

If the device's rotation is predominantly around a single axis, then the z-component of the rotated angular velocity signal will be of much greater magnitude than the components in the other two directions (the x- and y-axes). In this scenario the largest eigenvalue will be much greater than the other two eigenvalues. If, on the other hand, the device's axis of rotation is not constant, then the x and y components will generally be of greater magnitude. In this scenario the discrepancy in size between the largest eigen-value and the other two eigen-values will be less.

The ability of the subject to rotate the device around a single axis may be an indication of manual dexterity and thus a useful biomarker. Therefore, the ratio of the size of the largest eigenvalue to the size of one or both of the other two eigenvalues may be useful as an indicator of the presence and/or severity of a disease and/or disorder. This could be calculated in a number of ways, e.g. ratio of largest eigen-value to second largest eigen-value; or the ratio of largest eigen-value to the sum (or average) of the other two eigen-values; or the ratio of the energy in the z-component of the rotated angular velocity signal to the total energy in all 3 components of angular velocity, etc.

Once the data are available in the format in which they are aligned with the main axes, it is possible to convert the rotational velocity information into angular displacement information, as indicated at step 415-4. This task is also performed by the rotation angle processing unit 106-2-4. This allows the extraction of angular rotations as a function of time, separated into three orthogonal axes. The result of this applied to the data shown in FIG. 5B may be seen in FIG. 5C, discussed in more detail below.

As noted above, integration may be used to extract angular displacement from rotational velocity. However, for non-continuous signals (i.e. received at discrete times) a cumulative sim may be a preferred approach.

Using just the z-component of the rotated angular velocity data, and fₛ, the sampling rate, the cumulative sum of angular velocity is calculated to give the rotation angle at time tᵢ:

$$Φ ( {t}_{i} ) = {\sum }_{k = 1}^{i} {ω}_{z} ( {t}_{k} ) / {f}_{s}$$

The rotation angle can be used to calculate the range of movement of the body part, which is often a useful biomarker. Previous approaches have calculated the rotation angle, but have assumed that the device is aligned with the axis of rotation. If this is not the case, then the rotation angle calculated will be an underestimation and the range of movement and angular velocity of the body part will be inaccurate. This underestimation results in less accurate health measures, negatively impacting the ability for effective decision-making using these assessments.

Next, at step 415-5, the maxima and minima identification unit 106-2-5 is operable to identify the positions of the local maxima and minima of rotation angle Φ. These are identified in FIG. 5C with solid circles.

At step 415-6, the filtering unit 106-2-6 operates to exclude certain maxima and/or minima from the analysis. In FIG. 4, the criteria used are prominence criteria, but in other examples (additionally or alternatively) the criteria may be to discard a certain number of initial or final maxima and/or minima from the analysis.

Finally, the biomarker determination unit 106-2-7 is operable at steps 425 and 430 to extract one or more biomarkers from the data, by using the raw values of the parameters calculated, by calculating new parameters, by comparing parameter values to thresholds, by considering changes in parameter values throughout the duration of the data, and/or by combining parameters together.

It should be noted that while the above process 400 is described as being performed on the entire data set, in some cases the data may be divided into contiguous time blocks and each time block processed separately. For example a 20 second dataset may be subdivided into four 5 second blocks (or two 6 second blocks and an 8 second block) and each block then analysed separately according to the general method 400 set out above.

In some cases the time blocks may overlap one another, for example, the 20 second duration of an example test may be divided into a series of 9 overlapping blocks running from 0s to 4s, 2s to 6s, 4s to 8s, etc. These various options can be combined as needed to provide as much detail as desired regarding the change of parameters in different time blocks. Creating smaller subdivisions can be important to generate more accurate measures.

This approach may be particularly helpful for identifying changes in orientation of the primary axis. For example if a user finds it hard to hold their arm horizontally while making the motion shown in FIG. 3, dividing the data set into discrete contiguous time chunks may help to provide a nuanced view that this is what has happened, and allows a medical professional to distinguish between this example and e.g. a large off-axis “pitching” component to their test along with the correct model motion.

Moving on now to FIGS. 5A to 5C. As noted above these show three plots 500a to 500c respectively at various stages in the processing stream. In FIG. 5A the input data are shown in the form of angular velocity data in the format in which they were received from the device. This means that the data are aligned along the x-, y-, and z-axes of the device, irrespective of the motion which the user was trying to make. Even plotting the motion received from each axis of the sensor separately (as shown here), it is not immediately apparent whether the motion is a very messy rotation, or one in which the rotation is predominantly along a single axis.

In FIG. 5B, the plot 500b is of the same dataset, but with the data rotated to align the primary rotational axis (the eigenvector of the covariance matrix having the largest eigenvalue) with the z-axis. The secondary rotational axis (the eigenvector of the covariance matrix having the second largest eigenvalue) is aligned with the y-axis and the final, tertiary rotational axis is aligned with the x-axis. This provides much more clarity, since when the data are plotted along their individual axes (as shown here), it is clear that the rotation about the primary rotational axis is much greater than that along the other two axes. Also of note is that, even though the user was attempting to make a single axis rotation, the off axis components are not zero. This is particularly evident in the y-axis plot (as might be expected as it is the second strongest) between 2 and 4 seconds, for example.

In FIG. 5C, the angular displacement plot 500c is shown, with the maxima and minima highlighted with solid circles. Note that not all maxima and minima are highlighted in this way. I particular those which fail to meet some predefined prominence criteria are not highlighted, and in some cases will be excluded from the analysis.

The prominence criteria may require that angular motions exceed a specified prominence threshold, e.g. 60 degrees (1.04 radians). Similarly the local minima are filtered to only include those minima whose ‘negative’ prominence is above a corresponding threshold, i.e. the signal must rise by at least the threshold from the minimum before encountering a value lower than the minimum. As used herein, for a peak of height h to have prominence p, the signal must fall to a value of (h-p) or lower on both sides of that peak before encountering a value that is higher than h, or before encountering the first or last data points.

Using this model, it can be seen that the local maximum at 1.1 seconds does not satisfy the prominence criteria as the signal falls by less than the threshold before finding a higher maximum at 0.5 seconds. Likewise the maximum at 0.5 seconds does not satisfy the prominence criteria as the signal falls by less than the threshold before encountering the left-hand edge of the data.

When counting turns, writing Nₚ for the number of maxima after filtering by the prominence criteria and Nₙ for the number of minima after filtering by the prominence criteria, then the number of turns, where each turn consists of a first rotational motion in one direction (e.g. supination wherein the patient's hand moves from the back of the hand facing upward to the back of the hand facing downward by twisting the wrist) and a second rotational motion in the opposite direction to the first (e.g. pronation wherein the patient's hand moves from the back of the hand facing downward to the back of the hand facing upward by twisting the wrist), is

(Nₚ+Nₙ+1)/2.

Note that this may result in a half integer, representing a half turn. A half turn consists of a first rotation without a corresponding second rotation. e.g. a supination movement without the corresponding pronation movement; or a pronation movement without the corresponding supination movement.

The turn rate, the number of turns per unit of time, is defined as:

(Nₚ+Nₙ−1)/(2*(tₗₐₛₜ−t_{first}))

where t_{first} is the time of the earliest maximum or minimum, and t is the time of the latest maximum or minimum.

It can be seen in FIG. 5C that the latest maximum/minimum is not at the end of the sequence of turns, but half a turn earlier. Similarly the earliest maximum/minimum is not at the start of the sequence of turns, but half a turn later. The inventors have found that this results in more reliable turn rates compared to calculating the turn rate over the complete sequence of turns.

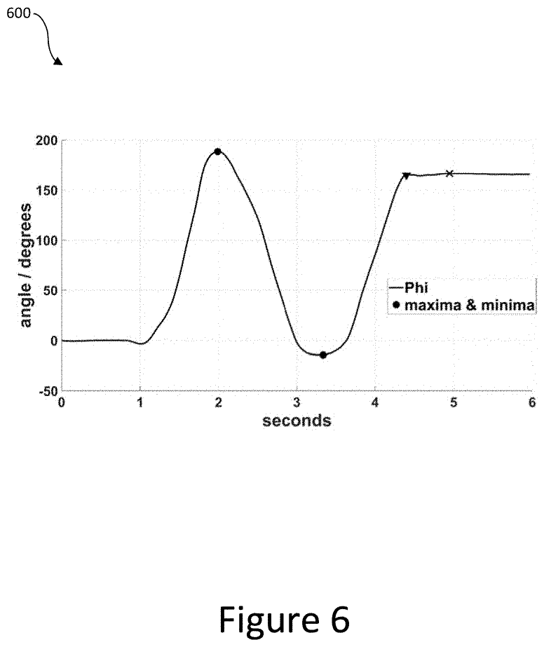

The reason behind this can be explained with reference to FIG. 6. FIG. 6 shows a short sequence 600 of one and a half turns. The maximum at 2.0 seconds and the minimum at 3.3 seconds are well defined. In contrast the end of the turns is harder to define—the peak value is at 5 seconds but as the signal is so flat at this point, a small amount of noise could easily shift the time of the peak. Indeed, a human eye would probably judge the turns to have finished earlier, at about 4.3 seconds. Thus, any estimate of the time of the end of turning is likely to be unreliable and can result in inaccurate extraction of biomarkers. Therefore, features depending on the duration of the sequence of turns, such as turn rate, are best calculated based on the time between the first and last maxima/minima, since the times of these are better determined than the start and end times of turning and this approach further improves the quality of the information provided by the method and apparatus discussed herein.

Finally, turning to FIGS. 7A and 7B, which show two histograms 700a, 700b of measurement data taken on a healthy population (FIG. 7A) and a population with a specific central nervous system disease (FIG. 7B). Here, the biomarker is provided as an axial variability parameter, Vₐₓ defined as:

$${V}_{ax} = 1 - \frac{max \{ {v}_{1} , {v}_{2} , {v}_{3} \}}{{\sum }_{i = 1}^{3} {v}_{i}}$$

in which v₁, v₂, and v₃ are the eigenvalues of the data about the primary, secondary and tertiary axes respectively.

Comparing the first histogram 700a with the second histogram 700b, it is clear that there is a distinction between the two data sets. Without any deep statistics being performed, it is clear that the healthy population is able to achieve scores lower on this measure than the diseased population can. This measure can therefore be used to generate biomarkers that may help distinguish between healthy subjects and subjects with neurodegenerative diseases, central nervous system disorders or other diseases impacting musculature control. Such a biomarker might also be useful for disease stratification.

In this example, Vₐₓ, the axial variability is a measure of the proportion of the rotation that is about axes other than the principal rotation axis (axis with the greatest eigenvalue, which is identified by the max{ } function). If all the rotation is about just one rotation axis, then Vₐₓ would be zero—the minimum possible value, which is why healthy patients score lower. On the other hand if there are equal amounts of rotation about all 3 axes (v₁=v₂=v₃), then Vₐₓ would be ⅔. In some cases, Vₐₓ may be scaled by a factor of 3/2 in order to make the measurement more intuitively run from 0 (perfect uniaxial motion) to 1 (no dominant axis at all).

Taking the data further, statistical analyses can assist in extracting useful information. For example, various averages can be extracted from the data as follows:

| | Mean | Mode | Median |
|---|---|---|---|
| Healthy | 0.014 | 0.010 | 0.012 |
| Diseased | 0.022 | 0.018 | 0.02 |

Note that while each average gives a different value in each case, there is a gap of about 0.008 between healthy and diseased in each measure. From these values, it may be seen that a value between a particular average for each population may be used to derive a threshold between healthy and diseased patients. In the above chart, a value of 0.014 or 0.016 may be used to categorise a patient as diseased or healthy. Of course a deeper statistical analysis of the populations may be provided to refine this finding. The value for the threshold, being derived from a specific dataset, may of course vary from disease to disease as different data will be collected specifically for each disease.

In addition, secondary measures may be used to disambiguate between different motions (and thereby may allow a clinician to distinguish between different diseases) by considering a supplementary measure. For example calculating v₁/v₂, or v₂/v₃ may to help identify if the off-axis motion is largely limited to one other axis or is more evenly distributed among the other two. This in turn can provide clues to the cause of the user's inability to correctly replicate the target motion.

Note that there is overlap between the Vₐₓ scores of the two groups so this biomarker may beneficially be combined with other biomarkers to produce a composite biomarker that, by combining multiple aspects of the disease, may be more reliable than any of the individual biomarkers.

While the two histograms 700a, 700b crudely divide the population into two groups (healthy and diseased respectively), further nuance can be provided by subdividing the diseased population into subgroups. As one example, where a recognized severity scale for a disease exists, the diseased population can be divided into different groups for each specific stage of the disease and a histogram plotted for each group. For example, where the disease is assessed on a five point scale, the population could be grouped into patients measuring 0 (healthy), 1, 2, 3, 4 and 5 on that scale. A threshold can then be determined between each grouping in line with the above analysis to allow a clinician to easily place patients into the appropriate severity level using the scale. Moreover, this nuanced approach may allow the clinician to provide a severity level of e.g. a 3.5 (or even more finely gained than this—any value between 0 and 5 to one or two decimal places) on the scale. This is a more nuanced analysis than is presently possible, and may allow a clinician to start treatment appropriate to an advanced severity level earlier than is currently possible. As is well-known, early interventions in diseases usually improves prognosis.

It will be appreciated that, while this example is presented in terms of the specific biomarker, Vₐₓ, the general principles set out above in which a biomarker is extracted and compared to a threshold apply equally to analysis of any biomarker extracted from the rotational motion data. As noted above, the threshold may be extracted from statistical analyses of that biomarker (or indeed of other, related biomarkers) across a population, for example examining correlations between the value for the biomarker and independent assessments of e.g. the severity of a particular disease or disorder.

### MODIFICATIONS AND VARIATIONS

A detailed embodiment has been described above. Various modifications and changes can be made to the above embodiment. Some of these variations will now be described.

In the examples above, it is assumed that the data are provided in rotational format, specifically rotational velocity. If angular velocity data is not available and if the axis of rotation lies predominantly in the horizontal plane, as it generally does for the hand pronation-supination assessment described above, then in an alternative implementation, angular velocity can be estimated from linear acceleration data. As the device rotates the direction of gravity, which will be the dominant contribution to the linear acceleration data, will also appear to rotate relative to the device and from this the component of the angular velocity in the horizontal plane may be calculated:

ω(tᵢ)=â(tᵢ₋₁)×â(tᵢ)*fₛ

where ω(tᵢ) is the estimated angular velocity vector at time tᵢ, â(tᵢ) is the linear acceleration unit vector, which includes gravitational acceleration, and × denotes the cross-product. Once more fₛ is the sampling frequency.

Compared to using angular velocity data as described above, this implementation has the disadvantage that it cannot detect the component of rotation about the vertical axis, and linear accelerations of the device may be misinterpreted as angular velocity.

Further, in the case that the user's device has multiple sensors built into it (e.g. more than one accelerometer and/or gyroscope), the data from each sensor may be analysed and the results combined (for example averaged) to work out more accurate or less noisy biomarkers. Similarly, where the user is carrying multiple devices (such as a cellular telephone) and an actigraph device, where both devices have a sensor, the system can extract biomarkers using the data from both sensors. The measurements from the two (or more) sensors can then be averaged again to improve signal to noise ratio or the measurements from one sensor may be used to corroborate or validate the biomarker(s) determined from rotational motion data obtained from the other sensor.

In the above embodiment, a software application for processing accelerometer data was provided in the user device. The same or similar software may be provided in the computer of the central server—so that the central server performs the above biomarker determination and extraction analysis. This software application may be provided as computer implementable instructions on a carrier signal or on a tangible computer readable medium. Alternatively, the functions of the software application may be defined in hardware circuits such as in FPGA or ASIC devices.

It will be appreciated from the above description that many features of the different examples are interchangeable and combinable. The disclosure extends to further examples comprising features from different examples combined together in ways not specifically mentioned. Indeed, there are many features presented in the above examples and it will be apparent to the skilled person that these may be advantageously combined with one another. In particular, features presented as part of a method may be included as part of an apparatus and vice-versa.

The application also includes the following numbered clauses that define various aspects of the invention:

1. A method of determining a medical biomarker indicative of a neurodegenerative disease or other disease impacting musculature control, the method comprising:

receiving motion data from a sensor held or mounted for rotation with a body part of the user during a repeated rotational motion of the body part, the motion data being provided relative to a frame of reference associated with the sensor;

processing the data to identify the orientation in the frame of reference associated with the sensor of primary, secondary, and tertiary axes of rotation for the motion of the body part of the user, wherein each of the primary, secondary and tertiary axes are mutually perpendicular to one another;

extracting the medical biomarker by analysing the rotational motion of the body part of the user about at least one of the primary, secondary and tertiary axes.

2. The method according to clause 1, wherein analysing the rotational motion of the body part of the user is performed in a continuous manner throughout the repeated motion.

3. The method according to clause 1 or clause 2, wherein analysing the rotational motion includes extracting the rotational motion along the first, second and/or third axes as a function of time.

4. The method according to clause 3, further comprising extracting local maxima and minima by identifying times at which peaks and troughs have a prominence having a magnitude greater than a predetermined threshold.

5. The method according to clause 4, wherein the analysing is performed only on motions having a prominence greater than the predetermined threshold.

6. The method according to any one of the preceding clauses, wherein the first and/or last motion is excluded from the analysing.

7. The method according to any one of the preceding clauses, wherein the medical biomarker is indicative of a discrepancy between the motion of the body part and a model motion which the user is instructed to make.

8. The method according to any one of the preceding clauses, wherein extracting the medical biomarker includes extracting one or more of:

frequency of repetition of the rotational motion;

time for a single rotational motion;

average time for a single rotational motion;

an angular amplitude of the rotational motion;

asymmetry of an angular amplitude of the rotational motion relative to a neutral point;

asymmetry of angular velocity or acceleration of the rotational movement; and/or a ratio of an angular amplitude of the rotational motion along one of the first second and third axes to an angular amplitude of the rotational motion along a different one of the first, second and third axes.

9. The method according to any one of the preceding clauses, wherein extracting the medical biomarker includes detecting a change in one or more of: frequency of repetition of the rotational motion;

time for a single rotational motion;

the orientation of one or more of the first, second and third axes in the frame of reference associated with the sensor;

an angular amplitude of the rotational motion;

asymmetry of an angular amplitude of the rotational motion relative to a neutral point;

asymmetry of angular velocity or acceleration of the rotational movement; and/or

a ratio of an angular amplitude of the rotational motion along one of the first second and third axes to an angular amplitude of the rotational motion along a different one of the first, second and third axes.

10. The method according to any one of the preceding clauses, wherein a time period for which the repeated rotational motion occurs is divided into two or more distinct sub-periods of time and wherein the analysing is performed individually on motion data received during one of the sub-periods of time.

11. The method according to any one of the preceding clauses, wherein the processing uses principal component analysis to identify the orientation of the first, second and third axes.

12. The method according to any one of the preceding clauses, wherein a low-pass filter is applied to the motion data prior to the identifying step being performed.

13. The method according to any one of the preceding clauses, wherein the repeated rotational motion is a pronation and supination of the hand or bending and straightening of a knee or elbow.

14. The method according to any one of the preceding clauses, further comprising calculating, based on the orientation of the primary, secondary and tertiary axes, a transform to align the received motion data with the primary, secondary and tertiary axes.

15. The method according to clause 14, further comprising applying the transform to the received motion data to extract the rotational motion of the user along each of the primary, secondary and tertiary axes.

16. A tangible computer readable medium comprising computer implementable instructions for causing a programmable computer device to carry out the steps of any of the preceding the method clauses.

17. An apparatus for determining a medical biomarker indicative of a neurodegenerative disease or other disease impacting musculature control, the apparatus comprising one or more processors and memory configured to:

receive motion data from a sensor held or mounted for rotation with the body part of a user during a repeated rotational motion of the body part, the motion data being provided relative to a frame of reference associated with the sensor;

process the data to identify the orientation in the frame of reference associated with the sensor of primary, secondary, and tertiary axes of rotation for the motion of the body part of the user, wherein each of the primary, secondary and tertiary axes are mutually perpendicular to one another;

extract the medical biomarker by analysing the rotational motion of the body part of the user about at least one of the primary, secondary and tertiary axes.

18. The apparatus according to clause 17 further including an accelerometer and/or a gyroscope for supplying the motion data.

19. The apparatus according to clause 17 or clause 18, wherein the one or more processors and memory is further configured to analyse the rotational motion of the body part of the user is performed in a continuous manner throughout the repeated motion.

20. The apparatus according to any one of clauses 17 to 19, wherein the one or more processors and memory is further configured to analyse the rotational motion to extract the rotational motion along the first, second and/or third axes as a function of time.

21. The apparatus according to clause 20, wherein the one or more processors and memory is further configured to extract local maxima and minima by identifying times at which peaks and troughs have a prominence having a magnitude greater than a predetermined threshold.

22. The apparatus according to clause 21, wherein the one or more processors and memory is further configured to analyse only motions having a prominence greater than the predetermined threshold.

23. The apparatus according to any one of clauses 17 to 22, wherein the one or more processors and memory is further configured to exclude the first and/or last motion from the analysis.

24. The method according to any one of clauses 17 to 23, wherein the medical biomarker is indicative of a discrepancy between the motion of the body part and a model motion which the user is instructed to make.

25. The apparatus according to any one of clauses 17 to 24, wherein, as part of extracting the medical biomarker, the one or more processors and memory is further configured to extract one or more of:

frequency of repletion of the rotational motion;

time for a single rotational motion;

average time for a single rotational motion;

an angular amplitude of the rotational motion;

asymmetry of an angular amplitude of the rotational motion relative to a neutral point;

asymmetry of angular velocity or acceleration of the rotational movement; and/or

a ratio of an angular amplitude of the rotational motion along one of the first second and third axes to an angular amplitude of the rotational motion along a different one of the first, second and third axes.

26. The apparatus according to any one of clauses 17 to 25, wherein, as part of extracting the medical biomarker, the one or more processors and memory is further configured to detect a change in one or more of:

frequency of repletion of the rotational motion;

time for a single rotational motion;

the orientation of one or more of the first, second and third axes in the frame of reference associated with the sensor;

an angular amplitude of the rotational motion;

asymmetry of an angular amplitude of the rotational motion relative to a neutral point;

asymmetry of angular velocity or acceleration of the rotational movement; and/or

a ratio of an angular amplitude of the rotational motion along one of the first second and third axes to an angular amplitude of the rotational motion along a different one of the first, second and third axes.

27. The apparatus according to any one of clauses 17 to 26, wherein the one or more processors and memory is further configured to divide a time period for which the repeated rotational motion occurs into two or more distinct sub-periods of time and to perform the analysing individually on motion data received during one of the sub-periods of time.

28. The apparatus according to any one of clauses 17 to 27, wherein the one or more processors and memory is further configured to use principal component analysis to identify the orientation of the first, second and third axes.

29. The apparatus according to any one of clauses 17 to 28, wherein the one or more processors and memory is further configured to apply a low-pass filter to the motion data prior to the performing the identifying.

30. The apparatus according to any one of clauses 17 to 29, wherein the repeated rotational motion is a pronation and supination of the hand or bending and straightening of a knee or elbow.

31. The apparatus according to any one of clauses 17 to 30, wherein the one or more processors and memory is further configured to calculate, based on the orientation of the primary, secondary and tertiary axes, a transform to align the received motion data with the primary, secondary and tertiary axes.

32. The apparatus according to clause 31, wherein the one or more processors and memory is further configured to apply the transform to the received motion data to extract the rotational motion of the user along each of the primary, secondary and tertiary axes.

33. The apparatus according to clause 18 or any clause dependent thereon, wherein the one or more processors and the memory are provided on a first device and wherein the gyroscope and/or accelerometer is provided on a second device located in a remote location relative to the first device; and wherein the first and second devices include cooperating communications units for communicating with one another.

34. An apparatus according to any one of clauses 17 to 33 forming part of a clinical trial system comprising a central computer that communicates with a plurality of user devices, each user device being arranged to collect motion data relating to repeated rotational motion of a body part of a user associated with the user device; and wherein the central computer or at least one user device comprises the apparatus according to claim 16 for determining a medical biomarker.

35. A tangible computer readable medium comprising computer implementable instructions for causing a programmable computer device to become configured as an apparatus according to any one of apparatus clauses 17 to 34.

36. The method or apparatus according to any one of the preceding clauses, wherein the biomarker includes an axial variability parameter, Vₐₓ defined as:

$${V}_{ax} = 1 - \frac{max \{ {v}_{1} , {v}_{2} , {v}_{3} \}}{{\sum }_{i = 1}^{3} {v}_{i}}$$

in which v₁, v₂, and v₃ are the eigenvalues of the data about the primary, secondary and tertiary axes respectively.

37. The method or apparatus according to any one of the preceding clauses, wherein extracting the biomarker further includes comparing the biomarker to thresholds derived from statistical analyses of the biomarker across a population.

1. A method of determining a medical biomarker indicative of a neurodegenerative disease or other disease impacting musculature control, the method comprising:
receiving motion data from a sensor held or mounted for rotation with a body part of a user during a repeated rotational motion of the body part, the motion data being provided relative to a frame of reference associated with the sensor;
processing the data to identify orientation in the frame of reference associated with the sensor of primary, secondary, and tertiary axes of rotation for the motion of the body part of the user, wherein each of the primary, secondary and tertiary axes are mutually perpendicular to one another;
extracting the medical biomarker by analyzing the rotational motion of the body part of the user about at least one of the primary, secondary and tertiary axes.

2. The method according to claim 1, wherein analyzing the rotational motion of the body part of the user is performed in a continuous or ongoing manner throughout the repeated motion.

3. The method according to claim 1, wherein analyzing the rotational motion includes extracting the rotational motion along the first, second and/or third axes as a function of time.

4. The method according to claim 3, further comprising extracting local maxima and minima by identifying times at which peaks and troughs have a prominence having a magnitude greater than a predetermined threshold.

5. The method according to claim 4, wherein the analyzing is performed only on motions having a prominence greater than the predetermined threshold.

6. The method according to claim 1, wherein the first and/or last motion is excluded from the analyzing.

7. The method according to claim 1, wherein the medical biomarker is indicative of a discrepancy between the motion of the body part and a model motion which the user is instructed to make.

8. The method according to claim 1, wherein extracting the medical biomarker includes extracting one or more of:
frequency of repetition of the rotational motion;
time for a single rotational motion;
average time for a single rotational motion;
an angular amplitude of the rotational motion;
asymmetry of an angular amplitude of the rotational motion relative to a neutral point;
asymmetry of angular velocity or acceleration of the rotational movement; and/or
a ratio of an angular amplitude of the rotational motion along one of the first second and third axes to an angular amplitude of the rotational motion along a different one of the first, second and third axes.

9. The method according to claim 1, wherein extracting the medical biomarker includes detecting a change in one or more of:
frequency of repetition of the rotational motion;
time for a single rotational motion;
the orientation of one or more of the first, second and third axes in the frame of reference associated with the sensor;
an angular amplitude of the rotational motion;
asymmetry of an angular amplitude of the rotational motion relative to a neutral point;
asymmetry of angular velocity or acceleration of the rotational movement; and/or
a ratio of an angular amplitude of the rotational motion along one of the first second and third axes to an angular amplitude of the rotational motion along a different one of the first, second and third axes.

10. The method according to claim 1, wherein a time period for which the repeated rotational motion occurs is divided into two or more distinct sub-periods of time and wherein the analyzing is performed individually on motion data received during one of the sub-periods of time.

11. The method according to claim 1, wherein the processing uses principal component analysis to identify the orientation of the first, second and third axes.

12. The method according to claim 1, wherein a low-pass filter is applied to the motion data prior to the identifying step being performed.

13. The method according to claim 1, wherein the repeated rotational motion is a pronation and supination of the hand or bending and straightening of a knee or elbow.

14. The method according to claim 1, further comprising calculating, based on the orientation of the primary, secondary and tertiary axes, a transform to align the received motion data with the primary, secondary and tertiary axes.

15. The method according to claim 14, further comprising applying the transform to the received motion data to extract the rotational motion of the user along each of the primary, secondary and tertiary axes.

16. The method according to claim 1, wherein the biomarker includes an axial variability parameter, Vₐₓ defined as:
$${V}_{ax} = 1 - \frac{max \{ {v}_{1} , {v}_{2} , {v}_{3} \}}{{\sum }_{i = 1}^{3} {v}_{i}}$$
in which v₁, v₂, and v₃ are the eigenvalues of the data about the primary, secondary and tertiary axes respectively.

17. The method according to claim 1, wherein extracting the biomarker further includes comparing the biomarker to thresholds derived from statistical analyses of the biomarker across a population.

18. An apparatus for determining a medical biomarker indicative of a neurodegenerative disease or other disease impacting musculature control, the apparatus comprising one or more processors and memory configured to:
receive motion data from a sensor held or mounted for rotation with the body part of a user during a repeated rotational motion of the body part, the motion data being provided relative to a frame of reference associated with the sensor;
process the data to identify orientation in the frame of reference associated with the sensor of primary, secondary, and tertiary axes of rotation for the motion of the body part of the user, wherein each of the primary, secondary and tertiary axes are mutually perpendicular to one another;
extract the medical biomarker by analyzing the rotational motion of the body part of the user about at least one of the primary, secondary and tertiary axes.

19. The apparatus according to claim 18 further including an accelerometer and/or a gyroscope for supplying the motion data.

20. The apparatus according to claim 18, wherein the one or more processors and memory are further configured to analyze the rotational motion of the body part of the user being performed in a continuous manner throughout the repeated motion.

21. The apparatus according to claim 18, wherein the one or more processors and memory are further configured to analyze the rotational motion to extract the rotational motion along the first, second and/or third axes as a function of time.

22. The apparatus according to claim 21, wherein the one or more processors and memory are further configured to extract local maxima and minima by identifying times at which peaks and troughs have a prominence having a magnitude greater than a predetermined threshold.

23. The apparatus according to claim 22, wherein the one or more processors and memory are further configured to analyze only motions having a prominence greater than the predetermined threshold.

24. The apparatus according to claim 18, wherein the one or more processors and memory are further configured to exclude the first and/or last motion from the analysis.

25. The method according to claim 18, wherein the medical biomarker is indicative of a discrepancy between the motion of the body part and a model motion which the user is instructed to make.

26. The apparatus according to claim 18, wherein, as part of extracting the medical biomarker, the one or more processors and memory are further configured to extract one or more of:
frequency of repetition of the rotational motion;
time for a single rotational motion;
average time for a single rotational motion;
an angular amplitude of the rotational motion;
asymmetry of an angular amplitude of the rotational motion relative to a neutral point;
asymmetry of angular velocity or acceleration of the rotational movement; and/or
a ratio of an angular amplitude of the rotational motion along one of the first second and third axes to an angular amplitude of the rotational motion along a different one of the first, second and third axes.

27. The apparatus according to claim 18, wherein, as part of extracting the medical biomarker, the one or more processors and memory are further configured to detect a change in one or more of:
frequency of repetition of the rotational motion;
time for a single rotational motion;
the orientation of one or more of the first, second and third axes in the frame of reference associated with the sensor;
an angular amplitude of the rotational motion;
asymmetry of an angular amplitude of the rotational motion relative to a neutral point;
asymmetry of angular velocity or acceleration of the rotational movement; and/or
a ratio of an angular amplitude of the rotational motion along one of the first second and third axes to an angular amplitude of the rotational motion along a different one of the first, second and third axes.

28. The apparatus according to claim 18, wherein the one or more processors and memory are further configured to divide a time period for which the repeated rotational motion occurs into two or more distinct sub-periods of time and to perform the analyzing individually on motion data received during one of the sub-periods of time.

29. The apparatus according to claim 18, wherein the one or more processors and memory are further configured to use principal component analysis to identify the orientation of the first, second and third axes.

30. The apparatus according to claim 18, wherein the one or more processors and memory are further configured to apply a low-pass filter to the motion data prior to the performing the identifying.

31. The apparatus according to claim 18, wherein the repeated rotational motion is a pronation and supination of the hand or bending and straightening of a knee or elbow.

32. The apparatus according to claim 18, wherein the one or more processors and memory are further configured to calculate, based on the orientation of the primary, secondary and tertiary axes, a transform to align the received motion data with the primary, secondary and tertiary axes.

33. The apparatus according to claim 32, wherein the one or more processors and memory are further configured to apply the transform to the received motion data to extract the rotational motion of the user along each of the primary, secondary and tertiary axes.

34. The apparatus according to claim 19, wherein the one or more processors and the memory are provided on a first device and wherein the gyroscope and/or accelerometer is provided on a second device located in a remote location relative to the first device; and wherein the first and second devices include cooperating communications units for communicating with one another.

35. An apparatus forming part of a clinical trial system comprising a central computer that communicates with a plurality of user devices, each user device being arranged to collect motion data relating to repeated rotational motion of a body part of a user associated with the user device; and wherein the central computer or at least one user device comprises the apparatus according to claim 18 for determining a medical biomarker.

36. The apparatus according to claim 18, wherein the biomarker includes an axial variability parameter, Vₐₓ defined as:
$${V}_{ax} = 1 - \frac{max \{ {v}_{1} , {v}_{2} , {v}_{3} \}}{{\sum }_{i = 1}^{3} {v}_{i}}$$
in which v₁, v₂, and v₃ are the eigenvalues of the data about the primary, secondary and tertiary axes respectively.

37. The apparatus according to claim 18, wherein extracting the biomarker further includes comparing the biomarker to thresholds derived from statistical analyses of the biomarker across a population.
